(19) United States
(12) Patent Application Publication
(10) Pub. No.: US 2025/0229077 A1
(43) Pub. Date: Jul. 17, 2025

(54) Interventional Blood Pump with Outlet Flow Guide Structure

(71) Applicant: ROCKETHEART TECHNOLOGY CO., LTD, Tianjin (CN)

(72) Inventors: Zhifu HAN, Tianjin (CN); Xian WANG, Tianjin (CN); Xuman ZHANG, Tianjin (CN); Hongbin MA, Tianjin (CN); Mingqian DING, Tianjin (CN); Chao WANG, Tianjin (CN); Guogang SONG, Tianjin (CN)

(73) Assignee: ROCKETHEART TECHNOLOGY CO., LTD, Tianjin (CN)

(21) Appl. No.: 18/854,755

(22) PCT Filed: Jun. 1, 2023
(86) PCT No.: PCT/CN2023/097790

§ 371 Date: Oct. 7, 2024

(30) Foreign Application Priority Data
Aug. 18, 2022 (CN) 202210992732.8
Aug. 18, 2022 (CN) 202222175608.0

(51) Int. Cl. A61M 60/812; A61M 60/13; A61M 60/174; A61M 60/221; A61M 60/416

(52) U.S. Cl. CPC A61M 60/812; A61M 60/13; A61M 60/174; A61M 60/221; A61M 60/416

(57) ABSTRACT

Provided is an interventional blood pump, including a pump body and a driving unit. The pump body includes an impeller, a blood flow catheter, a blood flow inlet structure, and a blood flow outlet structure which are in driving connection to the driving unit. The blood flow outlet structure includes an outlet housing, an outlet base, and a guide vane structure arranged on the outlet base and connected to the outlet housing. At least a part of the impeller is accommodated in the outlet housing. The guide vane structure is configured to be capable of converting a rotational motion of the blood flowing out of the impeller into a primarily axial motion.

### CROSS-REFERENCE TO RELATED APPLICATION(S)

The present disclosure is a national stage application of International Patent Application No. PCT/CN2023/097790, which is filed on Jun. 1, 2023. The International Patent Application claims the priority of Chinese Application No. 202222175608.0, filed in the Chinese Patent Office on Aug. 18, 2022, and entitled “Interventional Blood Pump”, claims the priority of Chinese Application No. 202210992732.8, filed in the Chinese Patent Office on Aug. 18, 2022, and entitled “Interventional Blood Pump with Outlet Flow Guide Structure”, the entire contents of which are herein incorporated by reference.

### TECHNICAL FIELD

The present application relates to the field of medical instruments, in particular to an interventional blood pump which can be percutaneously inserted into the blood vessel of a patient.

### BACKGROUND

An interventional catheter pump, also known as an interventional blood pump, is commonly used for treatment of high-risk percutaneous coronary intervention (PCI) to reduce the ventricular work and provide necessary circulatory support for cardiac recovery and early assessment of residual myocardial functions. The most mature and advanced interventional catheter pump currently available in the world is the Impella series researched and developed by AbioMed company. This type of blood pumping auxiliary device is guided into the heart of a patient through a blood vessel. During operation, a catheter pump inlet is arranged in the ventricle, a catheter pump outlet is arranged in the artery, and the blood is pumped from the ventricle into the artery, so as to ensure blood perfusion to the coronary artery and various organs throughout the body during the PCI surgery, and reduce the cardiac load. This type of catheter pump is generally composed of components such as a catheter, an impeller and a motor, and includes a blood inlet located in the ventricle and a blood outlet located in the artery, wherein the structure of the blood inlet affects the angle and direction of blood flow; the impeller is a main power component of a catheter blood pump, which directly affects the effect of blood delivery and the destructive effect on blood cells; and the function of the blood outlet is to discharge the blood pumped out by the impeller to the downstream side, which affects the stability and regularity of a blood flow field.

In an existing blood pump, an outlet structure is provided with elliptical hollowed-out holes directly on a metal housing at an impeller outlet, and the blood sucked into a catheter by an impeller directly disperses out of these holes, so that the flow is relatively chaotic, the dissipation of energy is caused to a certain extent, the suction effect of the impeller on the blood is weakened, and the blood dispersing in the artery disrupts the regular flow field form. In addition, in an existing blood pump, when the blood flows into a pump body from an inlet, due to the design of the inlet structure, the blood flow will have certain viscous flow and vortices, which hinders the flow or damages the blood to a certain extent.

### SUMMARY

One objective of some embodiments of the present disclosure is to solve the above technical problems.

Therefore, an embodiment of the present disclosure provides an interventional blood pump, including a pump body and a driving unit. The pump body includes an impeller, a blood flow catheter, a blood flow inlet structure, and a blood flow outlet structure which are in driving connection to the driving unit, wherein the blood flow outlet structure includes an outlet housing, an outlet base, and a guide vane structure arranged on the outlet base and connected to the outlet housing; at least a part of the impeller is accommodated in the outlet housing; and the guide vane structure is configured to be capable of converting a rotational motion of a blood flowing out of the impeller into a primarily axial motion. When the blood pump operates, due to the high-speed rotation of the impeller around the shaft, the blood pumped out by the impeller also rotates at a high speed with a relatively large circumferential component. According to the embodiment of the present disclosure, the blood flow outlet structure with the guide vane structure can collect the blood pumped out by the impeller and guide the flow direction of the blood to convert the rotational motion of the blood into a primarily axial motion, so as to effectively promote the regularity of a blood flow field and play a rectifying role, thereby avoiding the energy dissipation at the outlet caused by blood flow disorder and improving the working efficiency of the entire pump. Secondly, in the embodiment of the present disclosure, the guide vane structure is connected to the outlet housing and the outlet base, and the impeller is rotatably fixed in the outlet housing. The overall structure is stable and easy to manufacture. The “primarily axial motion” means that an axial component of the motion direction of the blood flowing out of the guide vane structure is greater than a circumferential component and a radial component, wherein the axial direction, circumferential direction and radial direction are all defined with the blood flow catheter as a reference system, and the axial direction refers to the direction around which the impeller rotates.

According to some embodiments of the present disclosure, in the above interventional blood pump, the guide vane structure includes at least two vanes, each of the at least two vanes is a bent and twisted vane or a straight vane, the each of the at least two vanes includes a vane tip and a vane root which are respectively connected to the outlet housing and the outlet base, as well as an inlet edge and an outlet edge which are connected to the vane tip and the vane root, and the inlet edge is located on an upstream side of the outlet edge in an axial direction. The “bent and twisted vane” means that vane shapes from the vane root to the vane tip are different, and along a vane height direction, the vane is twisted and the generatrix of the vane is also bent. This type of vane can better adapt to changes in blood flow direction in narrow spaces, thereby more accurately guiding the blood flow direction to approach the axial direction. The “straight vane” means that both the vane root and the vane tip extend along the axial direction. In some embodiments, the guide vane structure includes three to five vanes. If the number of vanes is too small, a good rectifying role can not be achieved; and if the number of vanes is too large, the local resistance is too large, which increases the risk of damaging blood cells.

According to some embodiments of the present disclosure, in the above interventional blood pump, the each vane is a bent and twisted vane, a vane root setting angle and a vane tip setting angle gradually increase along a blood flow direction respectively, the vane root setting angle and the vane tip setting angle are basically consistent with a flow angle of blood flowing out of the impeller from a corresponding position at an inlet of the guide vane structure respectively, and the vane root setting angle is close to 90° at an outlet of the guide vane structure to reduce the circulation of blood at the outlet. The “vane root setting angle” refers to an angle between the tangent direction of the vane root profile at a certain point on the vane root and the tangent direction of the circumferential curve passing through this point. Similarly, the “vane tip setting angle” refers to an angle between the tangent direction of the vane tip profile at a certain point on the vane tip and the tangent direction of the circumferential curve passing through this point. The “fluid flow angle” also refers to an angle between the blood flow direction at a certain point of blood flow and the tangent direction of the circumferential curve passing through this point. According to the present disclosure, at the inlet of the guide vane structure, the vane root setting angle and the vane tip setting angle are made to be basically consistent with the flow angle of the blood flow at the corresponding position respectively, that is, the bent and twisted vane is configured to follow the blood flow direction to avoid collision between the high-speed blood flowing out of the impeller and the vane, thereby avoiding damaging the structure of blood cells and causing hemolysis. Along the inlet to outlet direction of the guide vane structure, the angle of the vane gradually changes, and at the outlet of the guide vane structure, the vane root setting angle becomes close to 90°, such as 85° to 90°. As a result, the blood flow is guided to a primarily axial flow at the outlet of the guide vane structure, thereby reducing circulation and linear loss in a flow channel.

In some embodiments, in the above interventional blood pump, the vane tip setting angle is close to 90° at the outlet of the guide vane structure, which can better ensure that the blood flow is guided to a primarily axial flow at the outlet of the guide vane structure. However, in some implementations, due to the limitation of the axial size of the guide vane structure, the outlet setting angle of the vane tip cannot be close to 90°, because this will form a dead zone of blood flow, which is actually not conducive to the smooth flow of blood.

According to some embodiments of the present disclosure, in the above interventional blood pump, a thickness of the each of the at least two vanes ranges from 0.2 mm to 0.4 mm. The value range is obtained through multiple simulations and experiments. When the vane thickness is less than the range, the structural strength is insufficient, and the accuracy of the manufacturing process cannot be ensured. When the vane thickness is greater than the range, the vane cannot provide excellent hydrodynamic characteristics and may clog the flow channel, so that the blood outflow rate is increased, and the anti-hemolysis property is poor.

According to some embodiments of the present disclosure, in the above interventional blood pump, a proximal end of the blood flow inlet structure is fixed at a distal end of the blood flow catheter or a distal end of the outlet housing, a distal end of the blood flow inlet structure includes an inlet base fixed with a pigtail catheter, the blood flow inlet structure includes a bell-shaped flow guide cone arranged on the inlet base and a blood suction inlet extending from the inlet base to the proximal end of the blood flow inlet structure, a diameter of a distal end of the flow guide cone is greater than a diameter of a proximal end of the flow guide cone, and the flow guide cone includes a conical or concave flow guide surface connecting the distal end and the proximal end. In this implementation, the inventors replace a boss-shaped part in an existing inlet structure with a conical surface or a “bell-shaped” structure having a gentle radian, and the structure plays a flow guide role to ensure that the blood smoothly and stably flows into the catheter, reduce the impact on blood cells, ensure the integrity of blood cells, and also reduce the pressure loss of the blood in the catheter.

According to some embodiments of the present disclosure, in the above interventional blood pump, a rotational generatrix of the flow guide surface is a straight line, a concave circular or elliptical curve, in some embodiments, a concave elliptical curve is provided.

According to some embodiments of the present disclosure, in the above interventional blood pump, a diameter of an outer contour of the blood suction inlet is D and an axial length is H, the diameter of the proximal end of the flow guide cone is d1 and an axial length is h, and these parameters meet: 1.2 D≤H≤1.6 D, 0.3 D≤d1≤0.4 D, 0.4 D≤h≤0.7 D. By adjusting the axial length H of the blood suction inlet, a suitable cross-sectional flow area is obtained, thereby ensuring that the blood smoothly and stably flows into the catheter, and reducing the local resistance loss. By adjusting the contour parameter relationship of the flow guide cone, an excellent inlet flow guide effect is obtained, thereby controlling the blood to smoothly enter the catheter, reducing the impact on blood cells, and also reducing the pressure loss of the blood in the catheter.

According to some embodiments of the present disclosure, in the above interventional blood pump, the impeller is directly connected to an output shaft of the driving unit, the pump body sequentially includes the blood flow outlet structure, the impeller, the blood flow catheter, and the blood flow inlet structure from a proximal end to a distal end, the outlet base is fixed on a shell of the driving unit, and a proximal end of the blood flow catheter is fixed on the outlet housing. In the implementation where the impeller is directly connected to the output shaft of the driving unit, the driving unit is also located inside the body. The manners of connection between the parts of the pump body may be known in the art, such as bonding, threaded connection, laser welding, and integrated injection molding.

According to some embodiments of the present disclosure, in the above interventional blood pump, the impeller is connected to an output shaft of the driving unit through a flexible shaft, the pump body sequentially includes the blood flow catheter, the blood flow outlet structure, the impeller, and the blood flow inlet structure from a proximal end to a distal end, the blood flow catheter is a radially expandable catheter having a distal end connected to the outlet housing, and the blood flow catheter is arranged outside the flexible shaft and extends on at least a part of a length of the flexible shaft. When the blood flowing out of the blood flow outlet structure enters the radially expandable catheter and enables the radially expandable catheter to be in a radial expansion state, a gap between the radially expandable catheter and the flexible shaft forms a blood flow channel. A proximal part of the radially expandable catheter includes blood outlets located in an artery at a working position. In the implementation where the impeller is indirectly connected to the driving unit through the flexible shaft, the driving unit may be located inside the body or outside the body. In this implementation, the impeller is front-mounted to the distal end of the blood flow catheter and directly connected to the blood flow inlet structure, so that a blood pumping efficiency of the impeller can be improved. The blood flow outlet structure is arranged at a position adjacent to the impeller outlet, which can change the flow direction of the blood pumped out by the impeller to enable the blood to enter the radially expandable catheter mainly along the axial direction, thereby reducing the energy dissipation at the outlet caused by blood flow disorder and improving the working efficiency of the entire pump. During intervention, the radially expandable catheter can shrink to have a very small outer diameter to closely adhere to the flexible shaft, so that the radially expandable catheter can be inserted into the blood vessel conveniently. During the process of pumping blood, the radially expandable catheter can expand to have a relatively large inner diameter to form a blood flow channel having a relatively large cross section with the flexible shaft, thereby ensuring the flow area of the blood.

According to some embodiments of the present disclosure, in the above interventional blood pump, when the blood pump is located at the working position, both the driving unit and the pump body are located inside the body, and the proximal end of the radially expandable catheter is hermetically connected to an extending part at the distal end of the driving unit. The driving unit is arranged in the body, such as in the aorta or pulmonary artery, which can greatly shorten the length of the flexible shaft connecting the impeller and the driving unit, thereby avoiding the risk of wear, fracture and even damage to the blood vessel wall caused by the high-speed rotation of a long flexible shaft entering the blood vessel in a bent state, and also improving the transmission efficiency of the driving unit. However, the driving unit may also be located outside the body, and such technical solution already exists in the prior art. In such solution, there is no need to consider the size and heat dissipation of the driving unit. In addition, the radially expandable catheter belongs to a flexible structure which has relatively high instability. By connecting the proximal end of the radially expandable catheter to a rigid housing of the driving unit and connecting the distal end of the radially expandable catheter to an outlet shell of a rigid blood flow outlet structure, both ends are supported by rigid structures, which is beneficial for improving the stability.

In some embodiments, in the above interventional blood pump, the blood flow inlet structure and the blood flow outlet structure are both made of materials with good biocompatibility, such as implant grade metal materials or implant grade plastics.

It should be understood that the above general description and the following detailed description are merely exemplary and explanatory, but do not limit the present disclosure. Other features, objectives and advantages of the present disclosure will become apparent from the specification, drawings and claims.

### BRIEF DESCRIPTION OF THE DRAWINGS

In order to more clearly illustrate the technical solutions in the embodiments of the present application, the drawings required for description in the embodiments will be briefly introduced below. It is easy for those skilled in the art to understand that these drawings are only for illustrative purposes, but are not intended to limit the scope of protection of the present disclosure. For illustrative purposes, these drawings may not be completely drawn to scale.

FIG. 1 is a schematic overall structural view of an interventional blood pump according to an embodiment of the present application.

FIG. 2 is a partial cross-sectional view of a proximal part of the interventional blood pump shown in FIG. 1.

FIG. 3 is a three-dimensional schematic view of a blood flow outlet structure of the interventional blood pump shown in FIG. 1.

FIG. 4 is a three-dimensional schematic view of a flow guide structure of the blood flow outlet structure shown in FIG. 3.

FIG. 5 is a three-dimensional view of the flow guide structure shown in FIG. 4, wherein the entity of the guide vane structure is hidden, and only each vane root profile is retained.

FIG. 6 is a partial cross-sectional view of the interventional blood pump shown in FIG. 1.

FIG. 7 is an axial cross-sectional view of a blood flow inlet structure of the interventional blood pump shown in FIG. 1.

FIG. 8 is a schematic view of a flow guide cone of the blood flow inlet structure shown in FIG. 7.

FIG. 9 is a schematic overall structural view of an interventional blood pump according to another embodiment of the present application.

FIG. 10 is a three-dimensional schematic view of a blood flow outlet structure of the interventional blood pump shown in FIG. 9.

FIG. 11 is a partial cross-sectional view of a distal part of the interventional blood pump shown in FIG. 9.

### LIST OF REFERENCE NUMERALS

1: pigtail catheter;
2: blood flow inlet structure; 21: inlet base; 22: flow guide cone; 23: pigtail catheter connecting seat; 24: blood suction inlet; 25: guide wire through hole;
3: blood flow catheter; 30: radially expandable catheter; 302: blood outlet;
4: impeller; 42: impeller vane;
5: blood flow outlet structure; 52: outlet housing; 520: expanding part; 54: outlet base; 540: circumferential curve; 56: guide vane structure; 58: bearing;
6: driving unit; 61: output shaft;
7: flexible shaft; 72: soft shaft; 74: flat wire spring tube; 76: sealing flexible conduit; 78: impeller shaft;
80: vane; 81: inlet edge; 82: vane tip; 83: outlet edge; 84: vane root; 840: vane root profile; 85: connecting structure.

### DETAILED DESCRIPTION OF THE EMBODIMENTS

Exemplary implementations are described in detail herein, and examples of the exemplary implementations are shown in the drawings. When the following description involves the drawings, unless otherwise indicated, the same numerals in different drawings represent the same or similar elements. The implementations described in the following exemplary implementations do not represent all implementations consistent with the present application. On the contrary, they are merely examples of devices consistent with some aspects of the present application as described in the appended claims.

Unless otherwise defined, technical and scientific terms used herein have the same meanings as commonly understood by those skilled in the art of the present application. The terms used in the present application are for the purpose of describing specific embodiments only, and are not intended to limit the present application. The singular forms “a”, “the” and “this” used in the present application and the appended claims may also be intended to include plural forms, unless the contexts clearly indicate other meanings. It should also be understood that the term “and/or” used herein refers to and includes any or all possible combinations of one or more of the associated listed items. Terms such as “comprise”, “include” and the like mean that the components or objects appearing before “comprise” or “include” encompass the components or objects listed after “comprise” or “include” and equivalents thereof, without excluding other components or objects. Terms such as “connection”, “connected to”, and the like are not limited to physical or mechanical connections, and may include electrical connections, whether direct or indirect. “Multiple” includes two, equivalent to at least two. It should be understood that although the terms “first”, “second”, “third” and the like may be adopted in the present disclosure to describe various information, these information should not be limited to these terms. These terms are only used to distinguish the same type of information from each other. For example, the first information may also be referred to as the second information without departing from the scope of the present disclosure. Similarly, the second information may also be referred to as the first information.

In the present application, unless otherwise specified, the terms “proximal end” and “distal end” are relative to an operator of an interventional blood pump. The part of a component that is close to the operator is the proximal end, and the part that is far away from the operator is the distal end.

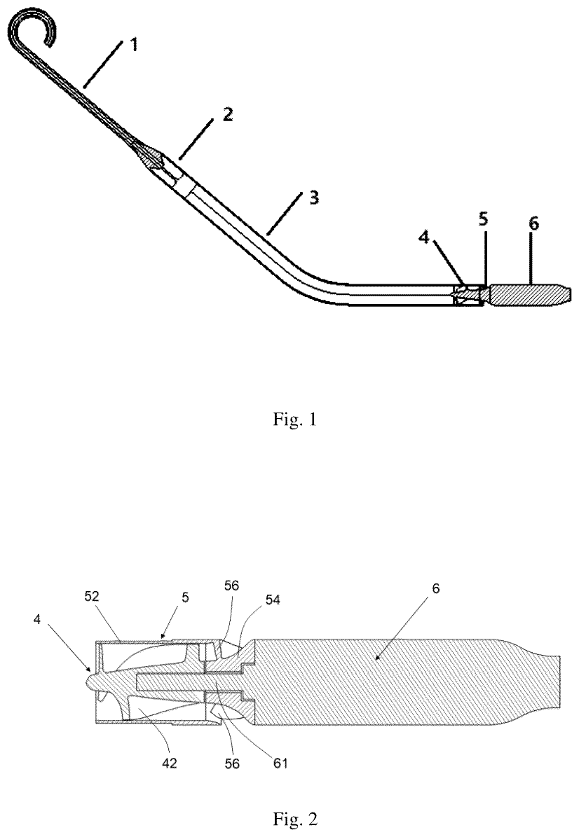

FIG. 1 schematically shows partial structures of an interventional blood pump according to an implementation of the present disclosure. The blood pump can serve as a ventricular assist device (VAD) to assist the ventricle in performing a blood pumping function during treatment of high-risk percutaneous coronary intervention. The blood pump includes a pump body located at a distal end and a driving unit 6 located at a proximal end during operation. In this embodiment, the driving unit 6 is a motor, such as a coreless motor. However, those skilled in the art should understand that any driving unit (such as a hydraulic motor) that can output power and is suitable for the field of interventional medicine can be used. The blood pump further includes a hollow interventional catheter (not shown), and a distal end of the interventional catheter is connected to a proximal end of the driving unit 6. During the surgical intervention process, the interventional catheter plays a pushing role.

In the embodiment shown in FIG. 1, from the distal end to the proximal end, the pump body sequentially includes a pigtail catheter 1, a blood flow inlet structure 2, a blood flow catheter 3, a rotatable impeller 4, and a blood flow outlet structure 5, wherein the impeller 4 is directly connected to an output shaft of the driving unit 6 and includes an impeller vane 42, the blood flow inlet structure 2 is provided with a blood suction inlet 24, and the blood pumped in by the impeller flows out of the blood flow outlet structure 5 and enters the artery. In some embodiments, a distal end of the pigtail catheter 1 is bent, which can prevent the pump body from touching the ventricular wall and causing unnecessary damage to the ventricular wall.

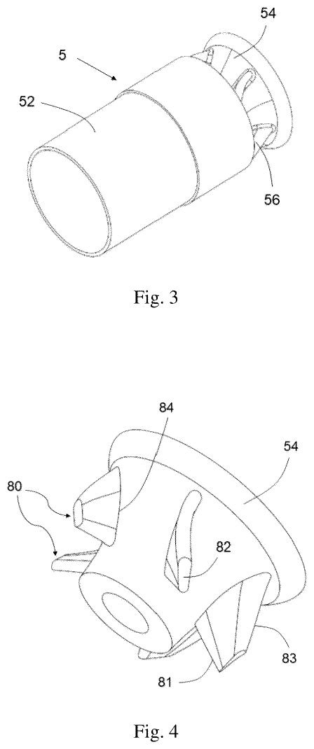

As shown more clearly in FIG. 2 and FIG. 3, the blood flow outlet structure 5 includes an outlet housing 52, an outlet base 54, and a guide vane structure 56 arranged on the outlet base 54 and connected to the outlet housing 52. A part of the impeller 4 is accommodated in the outlet housing 52. In this embodiment, the outlet base 54 is fixed on a shell of the driving unit 6, and a proximal end of the blood flow catheter 3 is fixed on the outlet housing 52. The connections between components can be made by various ways known and applicable in this field. For example, the blood flow catheter 3 is connected to the outlet housing 52 of the blood flow outlet structure 5 by adhesive bonding; the outlet housing 52 is connected to the driving unit 6 by laser welding; and the impeller 4 is connected to a driving motor by means of structural adhesive bonding, threaded connection or integrated injection molding. The present disclosure is not limited to this. When one component is fixed to another component, their assembly should have the feature of “axial alignment”.

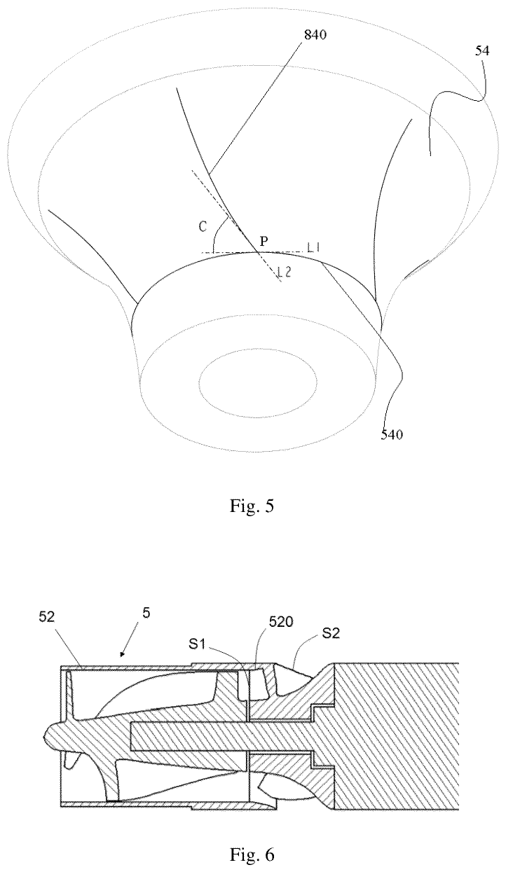

The guide vane structure 56 is configured to be capable of converting the rotational motion of the blood flowing out from the impeller 4 into a primarily axial motion. Therefore, in this embodiment, the guide vane structure 56 includes five vanes 80, each vane 80 has the same shape, and the vanes 80 are uniformly distributed along an axial direction. The thickness of the each vane 80 ranges from 0.2 mm to 0.4 mm. Each vane 80 includes a vane tip 82 connected to the outlet housing 52 and a vane root 84 connected to the outlet base 54, and the vane tip 82 and the vane root 84 are connected by an inlet edge 81 and an outlet edge 83. In the axial direction, the inlet edge 81 is located on an upstream side of the outlet edge 83. In some embodiments, as shown in FIG. 4, the vane 80 is a bent and twisted vane, that is, the vane is twisted along a vane height direction and the generatrix of the vane is also bent. For the convenience of showing a vane root setting angle C at the inlet, in FIG. 5, the entity of the guide vane structure 56 is hidden, only each vane root profile 840 is retained, and a circumferential curve 540 is shown. As shown in FIG. 5, at the starting point P of the vane root profile 840, namely the position corresponding to the most upstream side of the vane root 84, an angle C between a tangent direction L2 of the vane root profile 840 and a tangent direction L1 of the circumferential curve 540 passing through this point is the vane root setting angle at the inlet. By parity of reasoning, at any point on the vane root, the “vane root setting angle” refers to an angle between the tangent direction of the vane root profile at this point and the tangent direction of the circumferential curve passing through this point. Similarly, it can also be understood that the definition of the “vane tip setting angle” is the same as that of the “vane root setting angle”, which refers to an angle between the tangent direction of the vane tip profile at a certain point on the vane tip and the tangent direction of the circumferential curve passing through this point.

It should be understood that a high-speed rotation of the impeller 4 results in the blood pumped out by the impeller 4 having a very high rotation speed, and the shape of the impeller vane 42 results in different fluid flow angles at different radial positions of an impeller outlet. In order to reduce the damage to blood cells, at the inlet of the guide vane structure 56, the vane root setting angle and the vane tip setting angle are made to be basically consistent with the fluid flow angle of the blood flow at the corresponding position respectively, that is, the bent and twisted vane 80 is configured to follow the blood flow direction to avoid direct collision between the vane 80 and the high-speed blood flowing out from the impeller 4. Along the inlet to outlet direction of the guide vane structure 56, the angle of the vane 80 gradually changes, so that at the outlet of the guide vane structure 56, the vane root setting angle becomes close to 90°, such as 85° to 90°. As a result, the blood flow is guided to a primarily axial flow at the outlet of the guide vane structure 56, thereby reducing circulation and linear loss in a flow channel.

As a non-restrictive embodiment, an inlet setting angle of the vane tip ranges from 40° to 50°, an inlet setting angle of the vane root ranges from 51° to 61°, and an outlet setting angle of the vane tip ranges from 55° to 65°. When the outlet setting angle of the vane tip is also close to 90°, it can better ensure that the blood flow is guided to a primarily axial flow at the outlet of the guide vane structure. However, in the implementation shown in FIG. 3, due to the limitation of the axial size of the guide vane structure 56, an axial length of the vane tip 82 is relatively small. If the guide vane profile is forcibly twisted to enable the outlet setting angle of the vane tip 82 to be also close to 90°, a dead zone of blood flow will be formed, which is not conducive to smooth blood flow.

In addition, it is worth mentioning that, as shown in FIG. 6, the outlet housing 52 has an inner diameter variation near an expanding part 520 connected to the flow guide structure 56. Specifically, the inner diameter of the outlet housing 52 increases from the upstream side to the downstream side of the blood flow. This arrangement, together with the flow guide structure 56, gradually increases a flow area of the blood from S1 at the outlet of the impeller 4 to S2 at the outlet of the blood flow outlet structure 5 to form an expanding section, thereby converting part of the kinetic energy of the blood flow to pressure potential energy to play a role in stabilizing the blood flow.

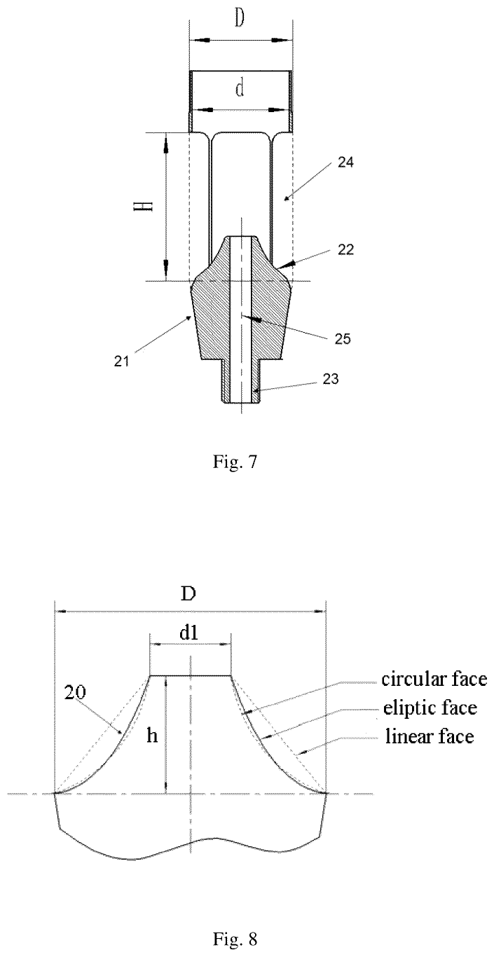

FIG. 7 is an axial cross-sectional view of a blood flow inlet structure. As shown in FIG. 7, in some embodiments, a distal end of the blood flow inlet structure 2 includes an inlet base 21, the inlet base 21 is fixed with a pigtail catheter 1. More specifically, a distal end of the inlet base 21 includes a pigtail catheter connecting seat 23 with a relatively small outer diameter, and the pigtail catheter 1 is fixed to the pigtail catheter connecting seat 23 by threads or adhesive bonding or threads and adhesive bonding. The inlet base 21 is also penetrated by a guide wire through hole 25 extending along the axial direction, which is used for enabling a guide wire to pass through to provide a guidance for the blood pump to enter the artery and heart. A bell-shaped flow guide cone 22 is formed at the connected part of the proximal end of the inlet base 21 and the blood suction inlet 24. It should be understood that the “bell-shaped” flow guide cone means that a three-dimensional shape likes a bell with both ends being different in size and connected by a smooth curve. Specifically, the diameter of the distal end of the flow guide cone 22 is greater than the diameter of the proximal end of the flow guide cone 22, and the distal end and the proximal end are connected by a smooth flow guide surface 20. As shown in FIG. 8, a rotational generatrix of the flow guide surface 20 may be a straight line, or a concave circular or elliptical curve, in some embodiments, a concave elliptical curve as shown in FIG. 7. As shown in FIG. 7, the diameter of the distal end of the flow guide cone 22 is the same as the diameter D of the outer contour of the blood flow inlet structure 2, the diameter of the proximal end is d1, and the axial length is h, wherein the diameter D of the outer contour is an input parameter of a catheter blood pump, which needs to be determined according to the application scope of a product and generally ranges from 4 mm to 7 mm. The inventors found that when the contour parameters of the flow guide cone 22 meet the following relationship: 0.3 D≤d1≤0.4 D, 0.4 D≤h≤0.7 D, a good guidance can be provided for blood flow, the blood can be controlled to smoothly enter the catheter, the impact of blood cells can be reduced, the integrity of blood cells can be ensured, and the pressure loss of the blood in the catheter can also be reduced. In addition, H represents an axial height of the blood suction inlet, which preferably meets 1.2 D≤H≤1.6 D to provide a good guidance for blood flow.

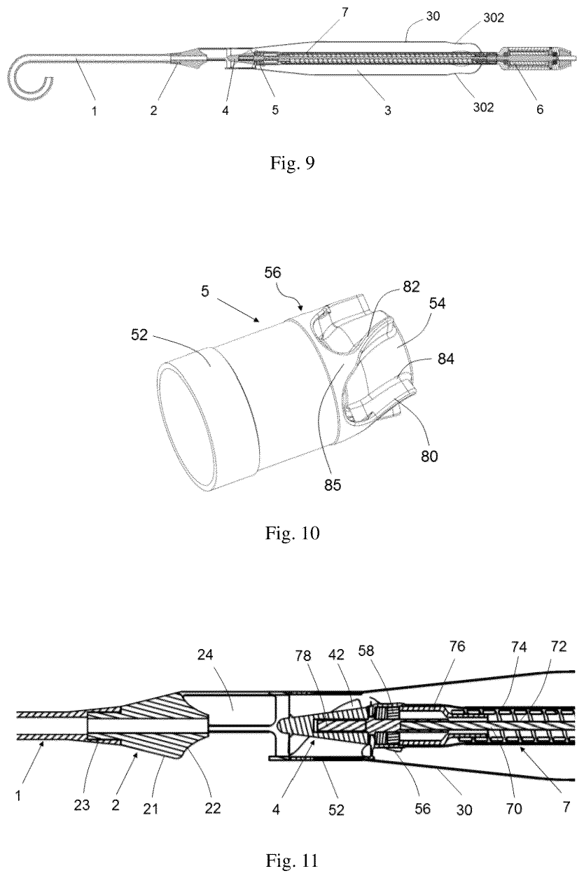

FIG. 9 shows an interventional blood pump according to another embodiment of the present application. Different from the previous embodiment, the impeller 4 is connected to the output shaft of the driving unit 6 through a flexible shaft 7. The flexible shaft 7 adopts a structure known in the art, including a soft shaft 70 for transmitting the torque of the driving unit 6 to the impeller 4 and driving the impeller 4 to rotate at a high speed, a flat wire spring tube 72 sleeved outside the soft shaft, and a sealing flexible conduit 74 sleeved outside the flat wire spring tube 72. It is worth noting that when the blood pump is located at a working position, the driving unit 6 may be located inside the body or outside the body. From the distal end to the proximal end, the pump body sequentially includes a pigtail catheter 1, a blood flow inlet structure 2, a rotatable impeller 4, a blood flow outlet structure 5, and a blood flow catheter 3, wherein the blood flow inlet structure 2 is provided with a blood suction inlet 24, the blood pumped in by the impeller 4 flows out of the blood flow outlet structure 5 and enters the blood flow catheter 3, the blood flow catheter 3 is formed by a radially expandable catheter 30 arranged outside the flexible shaft 7, a gap between the radially expandable catheter 30 and the flexible shaft 7 forms a blood flow channel, and a proximal part of the radially expandable catheter 30 includes blood outlets 302 located in the artery at a working position. Specifically, the distal end of the radially expandable catheter 30 may be fixed to an outer surface of the proximal end of the blood flow outlet structure 5 by means of adhesive bonding, hot melting, and the like. The blood outlets 302 are multiple openings formed on a proximal wall of the radially expandable catheter 30. The shape of the blood outlet 302 may be a circle, an ellipse, and the like. Three to six blood outlets 302 may be provided, and in some embodiments, the blood outlets 302 are uniformly distributed along a circumferential direction.

FIG. 10 schematically shows a three-dimensional view of the blood flow outlet structure 5 in the above embodiment. As shown in FIG. 10, the blood flow outlet structure 5 includes an outlet housing 52, an outlet base 54, and a guide vane structure 56 arranged on the outlet base 54 and connected to the outlet housing 52. The guide vane structure 56 includes five bent and twisted vanes 80, each vane 80 has the same shape, and the vanes 80 are uniformly distributed along an axial direction. Each vane 80 includes a vane tip 82 connected to the outlet housing 52 and a vane root 84 connected to the outlet base 54. The same as the embodiment shown in FIG. 3, at the inlet of the guide vane structure 56, the vane root setting angle and the vane tip setting angle are made to be basically consistent with the fluid flow angle of the blood flow at the corresponding position respectively, that is, the bent and twisted vane 80 is configured to follow the blood flow direction to avoid direct collision between the vane 80 and the high-speed blood flowing out of the impeller 4, thereby reducing the damage to blood cells. Along the inlet to outlet direction of the guide vane structure 56, the angle of the vane 80 gradually changes, so that at the outlet of the guide vane structure 56, the vane root setting angle becomes close to 90°, such as 85° to 90°. As a result, the blood flow is guided to a primarily axial flow at the outlet of the guide vane structure 56, thereby reducing circulation and linear loss in a flow channel. Different from the embodiment shown in FIG. 3, in the embodiment shown in FIG. 10, the vane tip setting angle at the outlet of the guide vane structure 56 is also close to 90°, which is more conducive to ensuring that the blood flow is guided to a primarily axial flow at different radial positions at the outlet of the guide vane structure. In addition, different from the embodiment shown in FIG. 3, in the embodiment shown in FIG. 10, the vane tip 82 is connected to the outlet housing 52 through a connecting structure 85 to obtain a stronger and more stable connection. The blood flow outlet structure 5 shown in FIG. 10 may be manufactured by machining.

In addition, different from the blood flow outlet structure shown in FIG. 6, the blood flow outlet structure 5 shown in FIG. 10 does not have an inner diameter variation, and there is no significant increase in the flow area of blood from the outlet of the impeller 4 to the outlet of the blood flow outlet structure 5. In fact, in this implementation, the expandable catheter 30 is used to increase the flow area so as to stabilize the blood flow.

Finally, as shown in an enlarged view in FIG. 11, in the implementation where the impeller 4 is connected to the driving unit 6 through the flexible shaft 7, the structural features and contour parameters of the blood flow inlet structure 2, especially the bell-shaped flow guide cone 22, are the same as or similar to those described in the previous embodiment, and will not be repeated here. The proximal end of the blood flow inlet structure 2 is directly connected to the outlet housing 52 of the blood flow outlet structure 5, and the impeller 4 is located between the blood flow inlet structure 2 and the blood flow outlet structure 5 and is mostly located in the outlet housing 52. A bearing 58 of an impeller shaft 78 is also arranged in the outlet base 54 of the blood flow outlet structure 5, and the impeller shaft 78 is rotatably supported in the bearing 58 and fixed to the distal end of the soft shaft 70. The fixation may be achieved in any suitable manner, such as bonding, laser welding, crimping or clamping. The pump body further includes a tubular connector 76 fixed to the proximal end of the blood flow outlet structure. The tubular connector 76 has a distal region and a proximal region. The outer diameter of the distal region is greater than the outer diameter of the proximal region, the distal region is connected to a distal end of the sealing flexible conduit 74, and the proximal region is connected to a distal end of the flat wire spring tube 72. In some embodiments, the tubular connector 76 is also made of a rigid material. In this way, the entire distal end of the flexible shaft 4 is connected to a rigid body, which can obtain good support and improve stability.

The drawings and the above descriptions describe the non-restrictive specific embodiments of the present application. In order to instruct the principle of the present disclosure, some conventional aspects have been simplified or omitted. Those skilled in the art should understand that any modifications, equivalent replacements, improvements, and the like made within the spirit and principle of the present application shall be included within the protection scope of the present application. Those skilled in the art should understand that the above features may be combined in various ways to form multiple variations of the present application in the case of no conflict. Therefore, the present disclosure is not limited to the above specific embodiments, but limited only by the claims and equivalents thereof.

1. An interventional blood pump, comprising a pump body and a driving unit, the pump body comprising an impeller, a blood flow catheter, a blood flow inlet structure, and a blood flow outlet structure which are in driving connection to the driving unit, wherein the blood flow outlet structure comprises an outlet housing, an outlet base, and a guide vane structure arranged on the outlet base and connected to the outlet housing; at least a part of the impeller is accommodated in the outlet housing; and the guide vane structure is configured to be capable of converting a rotational motion of a blood flowing out of the impeller into a primarily axial motion.

2. The interventional blood pump according to claim 1, wherein the guide vane structure comprises at least two vanes; each of the at least two vanes is a bent and twisted vane or a straight vane; the each of the at least two vanes comprises a vane tip and a vane root which are respectively connected to the outlet housing and the outlet base, as well as an inlet edge and an outlet edge which are connected to the vane tip and the vane root; and the inlet edge is located on an upstream side of the outlet edge in an axial direction.

3. The interventional blood pump according to claim 2, wherein the each vane is the bent and twisted vane; a vane root setting angle and a vane tip setting angle gradually increase along a blood flow direction respectively; the vane root setting angle and the vane tip setting angle are basically consistent with a flow angle of blood flowing out of the impeller from a corresponding position at an inlet of the guide vane structure respectively; and the vane root setting angle is close to 90° at an outlet of the guide vane structure to reduce the circulation of blood at the outlet.

4. The interventional blood pump according to claim 3, wherein the vane tip setting angle is close to 90° at the outlet of the guide vane structure.

5. The interventional blood pump according to claim 2, wherein a thickness of the each of the at least two vanes ranges from 0.2 mm to 0.4 mm.

6. The interventional blood pump according to claim 1, wherein a proximal end of the blood flow inlet structure is fixed at a distal end of the blood flow catheter or a distal end of the outlet housing; a distal end of the blood flow inlet structure comprises an inlet base fixed with a pigtail catheter; the blood flow inlet structure comprises a bell-shaped flow guide cone arranged on the inlet base and a blood suction inlet extending from the inlet base to the proximal end of the blood flow inlet structure; a diameter of a distal end of the flow guide cone is greater than a diameter of a proximal end of the flow guide cone; and the flow guide cone comprises a conical or concave flow guide surface connecting the distal end and the proximal end of the flow guide cone.

7. The interventional blood pump according to claim 6, wherein a rotational generatrix of the flow guide surface is a straight line, a concave circular curve or a concave elliptical curve.

8. The interventional blood pump according to claim 6, wherein a diameter of an outer contour of the blood suction inlet is D and an axial length is H, the diameter of the proximal end of the flow guide cone is d1 and an axial length is h, and these parameters meet: 1.2 D≤H≤1.6 D, 0.3 D≤d1≤0.4 D, 0.4 D≤h≤0.7 D.

9. The interventional blood pump according to claim 1, wherein the impeller is directly connected to an output shaft of the driving unit; the pump body sequentially comprises the blood flow outlet structure, the impeller, the blood flow catheter, and the blood flow inlet structure from a proximal end to a distal end; the outlet base is fixed on a shell of the driving unit; and a proximal end of the blood flow catheter is fixed on the outlet housing.

10. The interventional blood pump according to claim 1, wherein the impeller is connected to an output shaft of the driving unit through a flexible shaft; the pump body sequentially comprises the blood flow catheter, the blood flow outlet structure, the impeller, and the blood flow inlet structure from a proximal end to a distal end; the blood flow catheter is a radially expandable catheter having a distal end connected to the outlet housing; the blood flow catheter is arranged outside the flexible shaft and extends on at least a part of a length of the flexible shaft; when a blood flowing out of the blood flow outlet structure enters the radially expandable catheter, so that the radially expandable catheter is in a radial expansion state, a gap between the radially expandable catheter and the flexible shaft forms a blood flow channel; and a proximal part of the radially expandable catheter comprises blood outlets located in an artery at a working position.

11. The interventional blood pump according to claim 10, wherein when the blood pump is located at the working position, both the driving unit and the pump body are located inside a body, and the proximal end of the radially expandable catheter is hermetically connected to a housing of the driving unit.

12. The interventional blood pump according to claim 2, wherein a proximal end of the blood flow inlet structure is fixed at a distal end of the blood flow catheter or a distal end of the outlet housing; a distal end of the blood flow inlet structure comprises an inlet base fixed with a pigtail catheter; the blood flow inlet structure comprises a bell-shaped flow guide cone arranged on the inlet base and a blood suction inlet extending from the inlet base to the proximal end of the blood flow inlet structure; a diameter of a distal end of the flow guide cone is greater than a diameter of a proximal end of the flow guide cone; and the flow guide cone comprises a conical or concave flow guide surface connecting the distal end and the proximal end of the flow guide cone.

13. The interventional blood pump according to claim 3, wherein a proximal end of the blood flow inlet structure is fixed at a distal end of the blood flow catheter or a distal end of the outlet housing; a distal end of the blood flow inlet structure comprises an inlet base fixed with a pigtail catheter; the blood flow inlet structure comprises a bell-shaped flow guide cone arranged on the inlet base and a blood suction inlet extending from the inlet base to the proximal end of the blood flow inlet structure; a diameter of a distal end of the flow guide cone is greater than a diameter of a proximal end of the flow guide cone; and the flow guide cone comprises a conical or concave flow guide surface connecting the distal end and the proximal end of the flow guide cone.

14. The interventional blood pump according to claim 4, wherein a proximal end of the blood flow inlet structure is fixed at a distal end of the blood flow catheter or a distal end of the outlet housing; a distal end of the blood flow inlet structure comprises an inlet base fixed with a pigtail catheter; the blood flow inlet structure comprises a bell-shaped flow guide cone arranged on the inlet base and a blood suction inlet extending from the inlet base to the proximal end of the blood flow inlet structure; a diameter of a distal end of the flow guide cone is greater than a diameter of a proximal end of the flow guide cone; and the flow guide cone comprises a conical or concave flow guide surface connecting the distal end and the proximal end of the flow guide cone.

15. The interventional blood pump according to claim 5, wherein a proximal end of the blood flow inlet structure is fixed at a distal end of the blood flow catheter or a distal end of the outlet housing; a distal end of the blood flow inlet structure comprises an inlet base fixed with a pigtail catheter; the blood flow inlet structure comprises a bell-shaped flow guide cone arranged on the inlet base and a blood suction inlet extending from the inlet base to the proximal end of the blood flow inlet structure; a diameter of a distal end of the flow guide cone is greater than a diameter of a proximal end of the flow guide cone; and the flow guide cone comprises a conical or concave flow guide surface connecting the distal end and the proximal end of the flow guide cone.

16. The interventional blood pump according to claim 7, wherein a diameter of an outer contour of the blood suction inlet is D and an axial length is H, the diameter of the proximal end of the flow guide cone is d1 and an axial length is h, and these parameters meet: 1.2 D≤H≤1.6 D, 0.3 D≤d1≤0.4 D, 0.4 D≤h≤0.7 D.

17. The interventional blood pump according to claim 2, wherein the impeller is directly connected to an output shaft of the driving unit; the pump body sequentially comprises the blood flow outlet structure, the impeller, the blood flow catheter, and the blood flow inlet structure from a proximal end to a distal end; the outlet base is fixed on a shell of the driving unit; and a proximal end of the blood flow catheter is fixed on the outlet housing.

18. The interventional blood pump according to claim 3, wherein the impeller is directly connected to an output shaft of the driving unit; the pump body sequentially comprises the blood flow outlet structure, the impeller, the blood flow catheter, and the blood flow inlet structure from a proximal end to a distal end; the outlet base is fixed on a shell of the driving unit; and a proximal end of the blood flow catheter is fixed on the outlet housing.

19. The interventional blood pump according to claim 4, wherein the impeller is directly connected to an output shaft of the driving unit; the pump body sequentially comprises the blood flow outlet structure, the impeller, the blood flow catheter, and the blood flow inlet structure from a proximal end to a distal end; the outlet base is fixed on a shell of the driving unit; and a proximal end of the blood flow catheter is fixed on the outlet housing.

20. The interventional blood pump according to claim 5, wherein the impeller is directly connected to an output shaft of the driving unit; the pump body sequentially comprises the blood flow outlet structure, the impeller, the blood flow catheter, and the blood flow inlet structure from a proximal end to a distal end; the outlet base is fixed on a shell of the driving unit; and a proximal end of the blood flow catheter is fixed on the outlet housing.
